(19) United States
(12) Patent Application Publication
(10) Pub. No.: US 2018/0348228 A1
(43) Pub. Date: Dec. 6, 2018

(54) Method of Determining Quantity of Objects to be Detected in Specimen

(71) Applicants: Ortho-Clinical Diagnostics Kabushiki Kaisha, Tokyo (JP); Furukawa Electric Co., Ltd., Tokyo (JP)

(72) Inventors: Satoru Sugita, Chiba (JP); Masayuki Fukushima, Tokyo (JP); Masataka Nishida, Tokyo (JP); Michio Ohkubo, Tokyo (JP); Kazutomi Miyoshi, Tokyo (JP)

(21) Appl. No.: 15/779,006

(22) PCT Filed: Nov. 25, 2016
(86) PCT No.: PCT/JP2016/084946

§ 371 Date: May 24, 2018

(30) Foreign Application Priority Data
Nov. 26, 2015 (JP) 2015-230780

(51) Int. Cl. G01N 33/58; G01N 33/543; G01N 33/536

(52) U.S. Cl. CPC G01N 33/581; G01N 33/536; G01N 33/54326

(57) ABSTRACT

Provided is a method by which the quantity of objects to be detected can be quickly determined with high sensitivity. The method of determining the quantity of objects to be detected in a specimen comprises: a mixing step for preparing a mixture by mixing the specimen and carrier particles that carry stimulus-responsive substances and a first affinity substance for the objects to be detected; a measurement step for placing the mixture under the condition that the stimulus-responsive substances aggregate, and measuring the particle diameter of a suspended solid in the mixture; and a determination step for determining, on the basis of the particle diameter, the quantity of the objects to be detected.

### TECHNICAL FIELD

The present invention relates to a method of quantifying a detection target in a specimen.

### BACKGROUND ART

In the field of clinical laboratory tests and the like, antigen-antibody reactions and the like are used to detect a trace component in a specimen. Specimens include those obtained from a living body, for example, various body fluids such as serum, plasma, urine, and lymph fluid.

As a method of detecting a detection target in a specimen, Patent Document 1, for example, describes a method of detecting the presence of a detection target, the method including: mixing a first bound substance in which a stimuli-responsive substance is bound to a first affinity substance having an affinity for the detection target (and a second bound substance in which a second substance having an electric charge is bound to a second affinity substance having an affinity for the detection target) with a specimen; exposing the resulting mixture to aggregation conditions of the stimuli-responsive substance; and determining whether the stimuli-responsive substance is dispersed or not in the mixture by measuring the turbidity of the mixture. It also describes that the first bound substance (a magnetic substance) aggregated is subjected to solid-liquid separation through the use of magnetic force by arranging a magnet in a device for the purpose of increasing detection sensitivity.

In the above detection method, the stimuli-responsive substance experiences aggregation inhibition, and is thus dispersed when the detection target is present. In contract the stimuli-responsive substance undergoes aggregation when the detection target is not present. The presence or absence of such aggregation inhibition will be reflected in the turbidity of the mixture. For example, as shown in FIG. 3, the stimuli-responsive substance usually experiences increased aggregation inhibition and is dispersed as the amount of the detection target increases, showing decreased turbidity. Then, the relation between the amount of the detection target and the turbidity starts to be reversed, and the turbidity is eventually decreased below the initial value over time. This, which can be effected by solid-liquid separation of the aggregated magnetic substance by virtue of magnetic force, can provide higher detection sensitivity by using the difference between a measured value determined as the maximum value and a measured value at a time point after that.

Patent Document 1: PCT International Publication No. WO2008/001868

### DISCLOSURE OF THE INVENTION

### Problems to be Solved by the Invention

As described above, the method of detecting the presence of a detection target and quantifying the detection target, if any, based on the turbidity of a mixture requires a step of solid-liquid separation by virtue of magnetic force in order to obtain high detection sensitivity. This results in a prolonged detection time.

The present invention is made in view of the above circumstances. An object of the present invention is to provide a method capable of detecting and quantifying a detection target rapidly with high sensitivity.

### Means for Solving the Problems

The present inventors found that a detection target can be detected and quantified rapidly with high sensitivity by determining the degree of dispersion of a stimuli-responsive substance based on the particle size of a suspended substance in a mixture. Then the present invention has been completed. Specifically, the present invention can provide the followings.

(1) A method of quantifying a detection target in a specimen, the method including: a mixing step of mixing the specimen with a particulate carrier carrying a stimuli-responsive substance and a first affinity substance having an affinity for the detection target to prepare a mixture; a measurement step of measuring the particle size of a suspended substance in the mixture after exposing the mixture to aggregation conditions of the stimuli-responsive substance; and a determination step of determining the amount of the detection target based on the particle size.

(2) The method according to (1), which does not include a step of performing solid-liquid separation of an aggregate of the particulate carrier contained in the suspended substance prior to the determination step.

(3) The method according to (1) or (2), in which the particulate carrier includes a nonmagnetic substance.

(4) The method according to any one of (1) to (3), in which the measurement is performed by the dynamic light scattering method.

(5) The method according to (4), in which the particulate carrier has a mean refractive index of 1.3 or more.

(6) The method according to any one of (1) to (5), in which the measurement is performed prior to a time point at which the turbidity of a negative control reaches the maximum value after exposed to the aggregation conditions.

(7) The method according to any one of (1) to (6), in which a bound substance of a hydrophilic substance and a second affinity substance having an affinity for the detection target is further mixed at the mixing step in addition to the particulate carrier.

### Effects of the Invention

According to the present invention, a detection target can be quantified rapidly with high sensitivity.

### BRIEF DESCRIPTION OF THE DRAWINGS

FIG. 1 shows a graph representing the relationship between the measurement time (aggregation time) and the mean particle size, as determined by a quantification method according to one embodiment of the present invention.

FIG. 2 shows a graph representing the relationships between the measurement time (aggregation time) and the mean particle size, as determined by quantification methods according to Example and Comparative Example of the present invention.

FIG. 3 shows a graph representing the relationship between the measurement time (aggregation time) and absorbance, as determined by a conventional quantification method in which a detection target is qualified based on turbidity.

### PREFERRED MODE FOR CARRYING OUT THE INVENTION

Below, the embodiments of the present invention will be described, but the present invention shall not be limited to these.

The method of quantifying a detection target in a specimen according to an embodiment of the present invention includes: a mixing step of mixing the specimen with a particulate carrier (hereafter, may be referred to as a first bound substance) carrying a stimuli-responsive substance and a first affinity substance having an affinity for the detection target to prepare a mixture; a measurement step of measuring the particle size of a suspended substance in the mixture after exposing the mixture to aggregation conditions of the stimuli-responsive substance; and a determination step of determining the amount of the detection target based on the particle size.

First, the specimen, the detection target, and the first bound substance will be described. A second bound substance which is preferably used to achieve higher detection sensitivity will also be described.

### Specimen

Specimens include biological substances such as various body fluids, for example, serum, plasma, urine, and lymph fluid of human or animal, and feces; food and drink; tap water; and samples collected from environments of rivers and others.

### Detection Target

Targets which can be detected by the aforementioned detection method include, for example, environmental pollutants, food contaminants, and substances which can be used for clinical diagnoses. Specifically, such substances include dioxin; environmental hormones; agricultural chemicals; PCB (polychlorbiphenyl); organic mercury and others; prions; fungal toxins; fugu toxin; antibiotics; antifungal agents; human immunoglobulins G, M, A and E, human albumin, human fibrinogen (fibrins and degradation products thereof), α-fetoprotein (AFP), C-reactive protein (CRP), myoglobin, carcinoembryonic antigens, hepatitis virus antigens, human chorionic gonadotropin (hCG), human placental lactogen (HPL), HIV virus antigens, allergens, bacterial toxins, bacterial antigens, enzymes, hormones (for example, human thyroid stimulating hormone (TSH), insulin, and the like) contained in body fluid, urine, sputum, feces and the like, and drug agents, and the like.

### First Bound Substance

Particulate Carrier

There is no particular limitation for the particulate carrier as long as it is a suspensible substance to which a stimuli-responsive substance and/or a first affinity substance can be directly or indirectly bound. Examples of the particulate carrier include organic microparticles of silica, acrylic resins, and the like; and metal particles. Silica particles are preferred. Silica may be in the form of particles including silicon dioxide as the main component, or may be in the form of particles of substance commonly called silica, including particles of quartz or rock crystal. Silica particles which have silanol groups (Si—OH) on their particle surfaces, and are thus highly hydrophilic are preferred because they can easily be reacted in water. They are also preferred because they are non-toxic, easy to handle, and easily manufactured.

It is noted that in the present invention, the solid-liquid separation of an aggregate contained in a suspended substance by virtue of magnetic force needs not be performed as described below. Therefore, the particulate carrier does not need to be a magnetic substance which is attracted by magnetic force, but may be a nonmagnetic substance which does not affect a magnetic field.

The particulate carrier preferably has a mean refractive index of 1.3 or more, more preferably 1.3 or more to 4.0 or less, and more preferably 1.4 or more to 3.0 or less. The particulate carrier preferably has a mean refractive index falling within the above ranges because the particle size is preferably determined by a method of measurement based on scattering of measurement light (including diffraction, refraction, reflection, and absorption of light). It is noted that the mean refractive index is a value as measured at a wavelength of 589 nm and a temperature of 37° C. using an Abbe's refractometer (Model DR-A1, Atago Co., Ltd.) and a light source for spectroscopy.

Further, when the particle diameter of the particulate carrier is too large, suspensibility tends to be reduced. On the other hand, when it is too small, a property (that is, detection and quantification sensitivity) which may affect parameters such as a particle size tends to be deteriorated. The mean particle size of the particulate carrier may be suitably selected according to the above considerations, and it may be, for example, 0.05 to 1.0 μm, or may be 0.3 to 0.7 μm. The mean particle size may be a value as measured under a scanning electron microscope (SEM).

Stimuli-Responsive Substance

The stimuli-responsive substance can undergo a structural change in response to an external stimulus to control aggregation and dispersion. There is no particular limitation for the stimulus, and examples thereof include a change in temperature, irradiation of light, addition of an acid or a base (a change in pH), a change in an electric field, and the like.

In particular, the present invention may use a temperature-responsive polymer capable of undergoing aggregation or dispersion in response to a change in temperature as the stimuli-responsive substance. It is noted that temperature-responsive polymers include a polymer having a lower critical solution temperature (hereinafter may also be referred to as LCST), and a polymer having an upper critical solution temperature (hereinafter may also be referred to as UCST). For example, a polymer having a lower critical solution temperature (LCST) of 37° C. is completely dispersed in an aqueous solution at a temperature lower than LCST, and can undergo aggregation immediately after the water temperature is increased to LCST or higher. Further, a polymer having an upper critical solution temperature (UCST) of 5° C. is completely dispersed in an aqueous solution at a temperature higher than UCST, and can undergo aggregation immediately after the water temperature is decreased to UCST or lower.

Examples of a polymer having a lower critical solution temperature which may be used in the present invention include polymers of N-substituted (meth)acrylamide derivatives such as N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloylpiperidine, and N-methacryloylmorpholine; polyoxyethylene alkylamine derivatives such as hydroxypropylcellulose, partially acetylated polyvinyl alcohol, polyvinyl methyl ether, (polyoxyethylene-polyoxypropylene) block copolymer, and polyoxyethylene lauryl amine; polyoxyethylene sorbitan ester derivatives such as polyoxyethylene sorbitan laurate; (polyoxyethylene alkylphenyl ether) (meth)acrylates such as (polyoxyethylene nonylphenyl ether) acrylate and (polyoxyethylene octylphenyl ether) methacrylate; and polyoxyethylene (meth)acrylic acid ester derivatives, such as (polyoxyethylene alkyl ether) (meth)acrylates such as (polyoxyethylene lauryl ether) acrylate and (polyoxyethylene oleyl ether) methacrylate. Further, polymers thereof and copolymers of at least two of these monomers can also be used. Moreover, a copolymer of N-isopropylacrylamide and N-t-butylacrylamide can also be used. When a polymer including a (meth)acrylamide derivative is used, a different copolymerizable monomer may be copolymerized with that polymer in a range where the resulting product has a lower critical solution temperature. Among these, the followings may be preferably used in the present invention: a polymer composed of at least one monomer selected from the group consisting of N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloylpiperidine, and N-methacryloylmorpholine; or a copolymer of N-isopropylacrylamide and N-t-butylacrylamide. Further, an elastin-derived polypeptide having a repetitive sequence of a pentapolypeptide as exemplified by Val-Pro-Gly-X-Gly (wherein X is an amino acid other than proline), and others may also be used.

As a polymer having an upper critical solution temperature which can be used in the present invention, a polymer of at least one monomer selected from the group consisting of acryloylglycinamide, acryloylnipecotamide, acryloylasparaginamide, acryloylglutamineamide, and the like. Moreover, a copolymer of at least two of these monomers may also be used. A different copolymerizable monomer such as acrylamide, acetylacrylamide, biotinol acrylate, N-biotinyl-N′-methacryloyl trimethylene amide, acryloyl sarcosine amide, methacryl sarcosine amide, acryloyl methyl uracil may be copolymerized with these polymers in a range where the resulting product has an upper critical solution temperature.

A substance such as a pH-responsive polymer capable of undergoing aggregation and dispersion in response to a pH change may also be used as a stimuli-responsive substance in the present invention. There is no particular limitation for the value of pH where a pH-responsive substance undergoes a structural change, but it is preferably pH 4 to 10, more preferably pH 5 to 9 in view of preventing deteriorated detection/quantification accuracy which may be caused by denaturalization of a particulate carrier and a specimen upon applying a stimulus.

For such a pH-responsive polymer, polymers having a group such as carboxyl, phosphoric acid, sulfonyl, and amino as a functional group can be exemplified. More specifically, those may be used in which monomers having dissociable groups such as (meth)acrylic acid, maleic acid, styrenesulfonic acid, 2-acrylamido-2-methylpropanesulfonic acid, phosphorylethyl (meth)acrylate, aminoethyl methacrylate, aminopropyl (meth)acrylamide, and dimethylaminopropyl (meth)acrylamide may be polymerized. Or those may be used in which monomers having these dissociable groups may be copolymerized with other vinyl monomers, for example, (meth)acrylic acid esters such as methyl (meth)acrylate, ethyl (meth)acrylate, butyl (meth)acrylate; vinyl esters such as vinyl acetate and vinyl propionate; vinyl compounds such as styrene, vinyl chloride, and N-vinyl pyrrolidone; and (meth)acrylamides as long as pH-responsiveness is not impaired.

Moreover, the stimuli-responsive substance is not limited to the above stimuli-responsive polymers. It may be, for example, hydrogels disclosed in Japanese Patent No. 3693979, Japanese Patent No. 3916330, Japanese Unexamined Patent Application, Publication No. 2002-85957, Japanese Patent No. 4071738, Japanese Patent No. 2869684, Japanese Patent No. 2927601, Japanese Patent No. 3845249, and the like.

First Affinity Substance

There is no particular limitation for the first affinity substance as long as it has an affinity for a detection target. Here, the term “affinity” refers to a property in which a certain substance specifically binds to another substance. For example, when the detection target is an antigen, the first affinity substance may be an antibody against that antigen. When the detection target is an antibody, the first affinity substance may be an antigen for that antibody. For example, when the detection target is a GST-tagged protein, it may be glutathione. When the detection target is a histidine-tagged protein, it may be a chelating agent coordinated with a metal ion. When the detection target is a nucleic acid, it may be a nucleic acid having a complementary sequence.

The above antibody may be any type of immunoglobulin molecules, or may be a fragment of an immunoglobulin molecule having an antigen binding site such as Fab. The above antibody may also be a monoclonal antibody or a polyclonal antibody.

### Second Bound Substance

A second bound substance may be added at the mixing step described below in addition to the above first bound substance for the purpose of obtaining higher detection sensitivity. The above second bound substance has a configuration where a hydrophilic substance is bound to a second affinity substance having an affinity for a detection target.

Hydrophilic Substance

There is no particular limitation for the hydrophilic substance as long as it is a substance having hydrophilicity for an aqueous dispersion medium described below. Here, the term “hydrophilicity” refers to a property in which a certain substance has an affinity for an aqueous dispersion medium. Hydrophilic substances include, for example, hydrophilic polymer compounds having electric charges, and they are preferably polyanions or polycations. A polyanion means a substance having a plurality of anion groups, and a polycation means a substance having a plurality of cation groups. Polyanions include, for example, nucleic acids such as DNA and RNA. These nucleic acids have properties as polyanions because they each have a plurality of phosphodiester groups along the backbone of each nucleic acid. Further, polyanions also include polypeptides having many carboxylic acid functional groups (polypeptides including amino acids such as glutamic acid and aspartic acid); polymers including polyacrylic acid, polymethacrylic acid, acrylic acid, and/or methacrylic acid as polymerizable components; polysaccharides such as carboxymethylcellulose, hyaluronic acid, and heparin; and the like. Polycations include, for example, polylysine, polyarginine, polyornithine, polyalkylamine, polyethyleneimine, polypropylethyleneimine, and the like. It is noted that the number of functional groups on a polyanion (carboxyl group) or a polycation (amino group) is preferably 25 or more.

Second Affinity Substance

A substance may be used which can non-competitively bind to a detection target at a different site than the first affinity substance binds. For example, when the detection target is an antigen, the second affinity substance may be a monoclonal or polyclonal antibody which recognizes a different antigenic determinant of the detection target than the first affinity substance recognizes.

Below, a method of manufacturing the first bound substance will be described.

Method of Manufacturing First Bound Substance

The first bound substance may be prepared by allowing a stimuli-responsive substance and a first affinity substance to bind directly or indirectly with a particulate carrier. There is no particular limitation for the method of binding, but for example, a method of binding via a reactive functional group can be exemplified when a particulate carrier is bound directly to a stimuli-responsive substance. It can be performed by a method well-known in the art

Alternatively, binding of a stimuli-responsive substance and a first affinity substance (for example, a first antibody) may be achieved by, for example, attaching one of a pair of substances having an affinity for each other (for example, avidin and biotin; glutathione and glutathione S-transferase) to the stimuli-responsive substance and attaching the other to the first affinity substance, and allowing the stimuli-responsive substance to bind with the first affinity substance through these substances.

Specifically, as described in WO01/009141, attachment of biotin to a stimuli-responsive substance may be performed by attaching biotin or the like to a polymerizable functional group such as a methacrylic group and an acrylic group to obtain a addition-polymerizable monomer, which is then copolymerized with another monomer. Meanwhile, attachment of avidin or the like to a first affinity substance may be performed in accordance with a conventional method. Next, when the biotin-attached stimuli-responsive substance is mixed with the avidin-attached first affinity substance, the stimuli-responsive substance and the first affinity substance are bound through binding of avidin and biotin.

Alternatively, a method may be used including: copolymerizing a monomer having a functional group such as carboxyl, amino, or epoxy with another monomer when a substance such as a polymer is manufactured; and attaching a substance having an affinity for an antibody (for example, Melon® gel, protein A, protein G) to the resulting polymer through the functional group in accordance with a method well known in the art. The substance having an affinity for an antibody obtained as described above is allowed to bind with a first antibody to prepare a bound substance in which the stimuli-responsive substance is bound to the first antibody having an affinity for an antigen as a detection target.

Alternatively, a monomer having a functional group such as carboxyl, amino, or epoxy may be copolymerized with another monomer when a polymer is manufactured, and a first antibody having an affinity for an antigen as a detection target may be attached directly to the functional group in accordance with a conventional method.

Alternatively, the first affinity substance and the stimuli-responsive polymer may be bound to a particulate magnetic substance.

After the first affinity substance is exposed to conditions where the stimuli-responsive polymer aggregates, centrifugal separation may be performed to purify the first bound substance. The first bound substance may be purified by a method including attaching a stimuli-responsive polymer to a particulate magnetic substance, and further attaching a first affinity substance to the particulate magnetic substance, and then applying magnetic force to collect the particulate magnetic substance.

A particulate magnetic substance may be attached to a stimuli-responsive polymer by a method well known in the art such as a method in which attachment is mediated via a reactive functional group; a method in which a polymerizable unsaturated bond is introduced to an active hydrogen on polyhydric alcohol or polyhydric alcohol of the magnetic substance, and performing graft polymerization; and the like (for example, see ADV. Polym. Sci., vol. 4, p111, 1965 and J. Polymer Sci., Part-A, 3, p1031, 1965).

Preparation of Second Bound Substance

The second bound substance may be prepared by allowing a hydrophilic substance to bind directly or indirectly with a second affinity substance. There is no particular limitation for the method of binding, but for example, one of a pair of substances having an affinity for each other (for example, avidin and biotin; glutathione and glutathione S-transferase) is attached to a hydrophilic substance, and the other is attached to a second affinity substance (for example, a second antibody), and then the hydrophilic substance is allowed to bind indirectly with the second affinity substance through these substances.

For direct binding of a hydrophilic substance to a second affinity substance, they may be bound together through a functional group. For example, when a functional group is used, binding may be performed in accordance with the maleimide-thiol coupling method by Ghosh et al. (Ghosh et al., Bioconjugate Chem., 1, 71-76, 1990). Specifically, the following two methods can be exemplified.

According to a first method, first, a mercapto group (may also be referred to as a sulfhydryl group) is introduced into the 5′ end of a nucleic acid while 6-maleimide hexanoic acid succinimide ester (for example, “EMCS (product name)” (Dojindo Laboratories)) is allowed to react with an antibody to introduce a maleimide group. Next, these two substances are allowed to be bound together through the mercapto group and the maleimide group.

According to a second method, first, a mercapto group is introduced into the 5′ end of a nucleic acid as in the first method, and the mercapto group is further allowed to react with N,N-1,2-phenylenedimaleimide as a homo-bifunctional reagent to introduce a maleimide group into the 5′ end of the nucleic acid while a mercapto group is introduced into an antibody. Next, these two substances are allowed to be bound together through the mercapto group and the maleimide group.

In addition, as a method of introducing a nucleic acid into a protein, methods described in, for example, Nucleic Acids Research, 15; 5275 (1987), and Nucleic Acids Research, 16; 3671 (1988) are known. These technologies may be used for binding of a nucleic acid to an antibody.

According to Nucleic Acids Research, 16: 3671 (1988), first, an oligonucleotide is allowed to react with cystamine, carbodiimide, and 1-methylimidazole to introduce a mercapto group into the hydroxy group of the 5′ end of the oligonucleotide. The oligonucleotide into which the mercapto group has been introduced is purified, and then reduced with dithiothreitol. Subsequently, 2,2′-dipyridyl disulfide is added to introduce a pyridyl group into the 5′ end of the oligonucleotide through a disulfide bond. Meanwhile, iminothialene is allowed to react with a protein to introduce a mercapto group. The oligonucleotide into which the pyridyldisulfide group has been introduced is mixed with the protein into which the mercapto group has been introduced to allow a pyridyl group to specifically react with the mercapto group, thereby attaching the protein to the oligonucleotide.

According to Nucleic Acids Research, 15: 5275 (1987), first, an amino group is introduced into the 3′ end of an oligonucleotide, and dithio-bis-propionic acid-N-hydroxysuccinimide ester (may be abbreviated as dithio-bis-propionyl-NHS) as a homo-bifunctional reagent is then allowed to react. After the reaction, the disulfide bond in the dithio-bis-propionyl-NHS molecule is reduced by adding dithiothreitol, and a mercapto group is introduced into the 3′ end of the oligonucleotide. For treating a protein, a hetero-bifunctional cross-linking agent may be used as described in Japanese Unexamined Patent Application, Publication No. H5-48100. First, the hetero-bifunctional cross-linking agent which has a first reactive group (a succinimide group) capable of reacting with a functional group (for example, an amino group) in a protein and a second reactive group (for example, a maleimide group and others) capable of reacting with a mercapto group is allowed to react with the protein to introduce a second reactive group into the protein. This will be used as a pre-activated protein reagent. This protein reagent obtained as described above is covalently bonded with the mercapto group of the thiolated polynucleotide.

Below, a method of quantifying a detection target in a specimen using the aforementioned first bound substance will be described.

### Method of Quantification

The method of quantifying a detection target according to an embodiment of the present invention includes: a mixing step of mixing a particulate carrier (first bound substance) carrying a stimuli-responsive substance and a first affinity substance having an affinity for the detection target with a specimen to prepare a mixture; a measurement step of measuring the particle size of a suspended substance in the mixture after exposing the mixture to aggregation conditions of the stimuli-responsive substance; and a determination step of determining the amount of the detection target based on the particle size.

Mixing Step

First, the first bound substance is mixed with a specimen in a container to prepare a mixture. When higher detection sensitivity is required, a second bound substance is also preferably mixed together. They may be dispersed in an aqueous dispersion medium when preparing the mixture, if required. There is no particular limitation for the aqueous dispersion medium as long as it does not absorb laser at a wavelength used in an instrument for measuring dynamic light scattering as described below, and does not affect dissolution, swelling, and the like of the first bound substance, and has a refractive index different from those of the first bound substance and the second bound substance. For example, dispersion media include tris-hydrochloric acid buffer, phosphate buffer, borate buffer, and the like.

Measurement Step

The above mixture is exposed to aggregation conditions where the stimuli-responsive substance undergoes aggregation. When the detection target is present, the stimuli-responsive substance experiences aggregation inhibition and remains dispersed due to the presence of a charged moiety or a hydrophilic moiety of the detection target. In contrast, when the detection target is not present, the stimuli-responsive substance does not experience aggregation inhibition, and thus undergoes aggregation.

For example, when a temperature-responsive polymer is used as the stimuli-responsive substance, a container containing the mixture may simply be transferred to an incubator at a temperature where the temperature-responsive polymer undergoes aggregation. There are two types of temperature-responsive polymers: a polymer having an upper critical solution temperature (hereinafter may also be abbreviated as “UCST”) and a polymer having a lower critical solution temperature (hereinafter may also be abbreviated as “LCST”). For example, when a polymer having a lower critical solution temperature (LCST) of 37° C. is used, a container containing the liquid mixture may be transferred to an incubator at 37° C. or more to allow the temperature-responsive polymer to aggregate. When a polymer having an upper critical solution temperature (UCST) of 5° C. is used, a container containing the liquid mixture may be transferred to an incubator at less than 5° C. to allow the temperature-responsive polymer to aggregate.

It is noted that the aggregation of a temperature-responsive polymer may be performed before or simultaneously in parallel with binding of the first bound substance to the detection target, but the latter is preferred in view of a shortened treatment time.

Here, the lower critical solution temperature and the upper critical solution temperature can be determined, for example, as follows. First, a sample is transferred into a cell in an absorption spectrometer, and then heated at a rate of 1° C./minute. During this period, changes in transmittance at 550 nm are recorded. Here, a value of transmittance when a polymer is dissolved and transparent is considered as 100%, and a value of transmittance when the polymer is completely aggregated is considered as 0%. Then, a temperature at which transmittance is 50% is defined as LCST.

Further, when a pH-responsive polymer is used as the stimuli-responsive substance, an acid solution or an alkali solution may simply be added to a container containing the mixture. Specifically, an acid solution or an alkali solution may simply be added to a container containing a mixture having a pH outside the pH range where the pH-responsive polymer undergoes a structural change to change a pH of the content of the container to a pH within the pH range where the pH-responsive polymer undergoes a structural change. For example, when a pH-responsive polymer undergoing aggregation at pH 5 or less and dispersion above pH 5 is used, an acid solution may simply be added to a container containing a mixture dispersed at above pH 5 so that the pH becomes 5 or less. Alternatively, when a pH-responsive polymer undergoing aggregation at pH 10 or more and dispersion below pH 10 is used, an alkali solution may simply be added to a container containing a mixture dispersed below pH 10 so that the pH becomes 10 or more. There is no particular limitation for a pH where a pH-responsive polymer undergoes a structural change, but it is preferably pH 4 to 10, more preferably pH 5 to 9.

Alternatively, when a photo-responsive polymer is used, a container containing a liquid mixture may simply be irradiated with light having a wavelength capable of allowing the photo-responsive polymer to aggregate. A preferred type of light for initiating aggregation may vary depending on the type and structure of a photo-responsive functional group included in the photo-responsive polymer, but in general, ultraviolet or visible light having a wavelength of 190 to 800 nm may be used conveniently. In that case, the intensity is preferably 0.1 to 1000 mW/cm². It is noted that in view of increased measurement accuracy, a photo-responsive polymer preferably will not readily undergo dispersion, in other words, will remain aggregated upon irritation with light for measuring the particle size. When a photo-responsive polymer is used which undergoes dispersion upon irritation with light for measuring the particle size, a shortened irritation time can increase measurement accuracy.

The particle size of a suspended substance in a mixture can be measured with a commercially available particle size distribution measuring device and the like. As methods of measuring particle size distribution, known are the dynamic light scattering method, light microscopy, confocal laser scanning microscopy, electron microscopy, atomic force microscopy, the static light scattering method, laser diffractometry, the centrifugal sedimentation method, the electric pulse measurement method, chromatography, the ultrasonic attenuation method.

In the present invention, a particle size distribution measuring device by the dynamic light scattering method (DLS) is preferably used in view of the particle size range and convenient measurement. The particle size distribution measuring device by the dynamic light scattering method (DLS) may be of a discrete type, but a discrete-type measuring device is not preferred in view of continuous and smooth measurement of the particle size. It is noted that the term “particle size by the dynamic light scattering method” encompasses a concept including not only primary particles but also secondary particles composed of aggregated primary particles, which are measured by the dynamic light scattering method, and represents a measure for evaluating the degree of dispersion of a complex in which a stimuli-responsive substance of a first bound substance is aggregated. Commercially available measuring devices in which the dynamic light scattering method is used include a particle size distribution measuring device by the dynamic light scattering method [product name: Malvarn ZETA SIZER Nano-ZS] and the like.

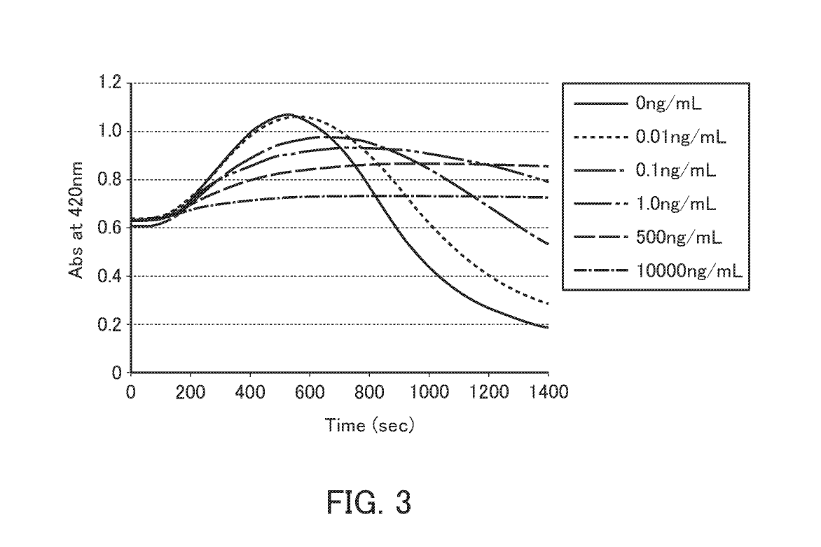

Measurement time (aggregation time) for the particle size of a suspended substance can appropriately be selected, considering required rapidity and sensitivity. When rapidity is required, measurement may be performed prior to a time point at which the turbidity (absorbance) of a solution containing no detection target reaches the maximum value after exposed to aggregation conditions. For a method of quantifying a detection target by measuring a particle size, significantly large changes in the particle size may become apparent in a very early stage under aggregation conditions. Therefore, in an embodiment of the present invention, a detection target can be detected with high detection sensitivity, for example, prior to a time point of 600 seconds at which the turbidity (absorbance) of a (zero) solution containing no detection target reaches the maximum value as shown in FIG. 3. On the other hand, when higher sensitivity is required, measurement may continue even after the aforementioned time point (a time required for the turbidity (absorbance) of a solution containing no detection target to reach the maximum value after exposed to aggregation conditions).

It is noted that a specimen (blood and others) suspected of including a detection target often abundantly contains various impurities, but the particle size to be measured will not significantly be affected by these impurities contained together with the detection target. For this reason, a preliminary step of removing impurities before measurement and other similar steps may not necessarily be performed.

Determination Step

Correlation Equation

A correlation equation for relating the amount of a detection target with the particle size of a suspended substance is created under the same conditions used at the above measurement step. A more reliable correlation equation can be obtained when a larger amount of data is available from measurements of the amount of a detection target and the particle size of a suspended substance for the correlation equation. Here, the above data preferably includes 2 or more pieces of information about the amount of a detection target, preferably 3 or more pieces of information about the amount of a detection target.

Calculation

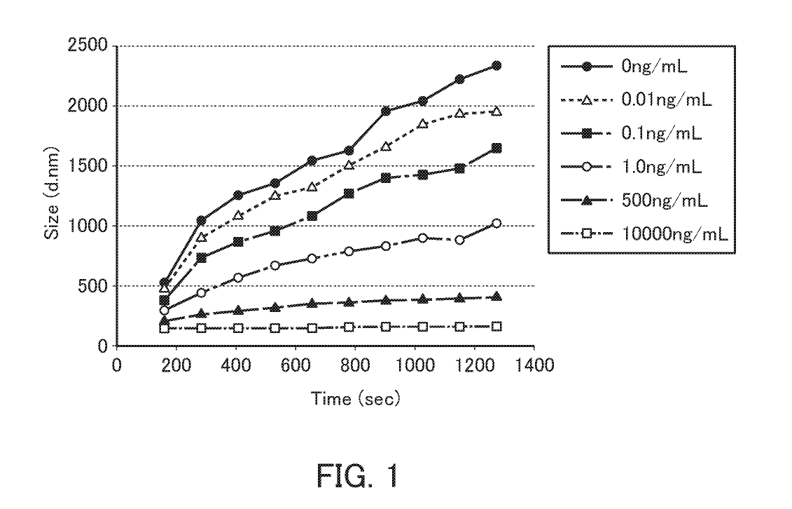

A measured value of the particle size of a suspended substance obtained from the aforementioned measurement step may be substituted into a correction equation created as described above to calculate the amount of a detection target in a specimen. For example, when a polymer having a lower critical solution temperature (LCST) of 37° C. is used as a stimuli-responsive substance, the particle size may change as shown in FIG. 1 depending on the concentrations of an antigen when transferring a container containing a liquid mixture to an incubator at 37° C. or more. It is noted that the results shown in FIG. 1 represent measurements performed under the same conditions as used at the measurement step described in Example 1 below except that the concentrations of a biotin-labelled anti-TSH beta antibody are different.

Functional Effect

When a first bound substance is mixed with a detection target, a first affinity substance binds to the detection target, if any, such that a charged moiety or a hydrophilic moiety of the detection target approaches a stimuli-responsive substance bound to the first affinity substance. This brings the charged moiety or the hydrophilic moiety to the proximity of the stimuli-responsive substance, which in turn inhibits the aggregation of the stimuli-responsive substance in response to a stimulus. In contrast, when the detection target is not present, the stimuli-responsive substance does not experience aggregation inhibition, and thus undergoes aggregation. Therefore, a smaller amount of a detection target would promote aggregation of a stimuli-responsive substance in a mixture over time to increase the particle size of a suspended substance over time. As understood from comparison of FIG. 1 with FIG. 3, a method of quantifying a detection target based on the particle size of a suspended substance can provide higher detection sensitivity even when the concentration of a detection target is small. This is because changes in the particle size become apparent in an earlier stage as compared with a method of quantifying a detection target based on the turbidity of a suspended substance. Further, a step of performing solid-liquid separation of an aggregate of a particulate carrier (first bound substance) is not necessarily required before the determination step as required when a detection target is quantified based on the turbidity of a suspended substance. Therefore, measurement time can be significantly shortened.

Further, when a first bound substance and a second bound substance are mixed with a detection target, a first affinity substance and a second affinity substance bind to the detection target, if any, such that a charged moiety or a hydrophilic moiety of the detection target approaches a particulate carrier or a stimuli-responsive substance bound to the first affinity substance. Or the charged moiety or the hydrophilic moiety bound to the second affinity substance approaches the particulate carrier or the stimuli-responsive substance bound to the first affinity substance. This brings the charged moiety or the hydrophilic moiety to the proximity of the stimuli-responsive substance, which in turn inhibits aggregation of the stimuli-responsive substance in response to a stimulus. In contrast, when the detection target is not present, the stimuli-responsive substance does not experience aggregation inhibition, and thus undergoes aggregation. Therefore, a smaller amount of a detection target would promote aggregation of a stimuli-responsive substance in a mixture over time to increase the particle size of a suspended substance over time. Thus, when a second bound substance is used in combination, the magnitude of changes, which depends on the amount of a detection target, is larger as compared with a case where the first bound substance is mixed with the detection target. Therefore, when the amount of a detection target is very small, and thus high detection sensitivity is required, the second bound substance may be further added at the mixing step in addition to the first bound substance.

As described above, in the method of quantifying a detection target based on the particle size of a suspended substance, the detection target can be detected more rapidly with higher sensitivity as compared with a case where the detection target is detected based on the turbidity of a suspended substance.

### EXAMPLE

### Preparation of First Bound Substance

First, an antibody (clone: Mouse 195, mouse IgG, Leinco Technology, Inc.), which is a first affinity substance, against human thyroid stimulating hormone (TSH), which is a detection target, was biotinylated by the well-known conventional sulfo-NHS-biotin method (AGC Techno Glass Co., Ltd.) to prepare a biotin-labelled anti-TSH beta antibody.

Meanwhile, 250 μL of Magnabeat Therma-Max LSA Streptavidin (0.4 mass %) as a streptavidin-attached particulate magnetic substance was transferred into a 1.5 mL micro tube. The Therma-Max LSA Streptavidin was allowed to aggregate by heating the micro tube to 42° C., and then magnetically collected. The supernatant was removed. To this, added was 250 pL of TBS buffer (20 mM Tris-HCl, 150 mM NaCl, pH 7.5). Aggregates were then dispersed by cooling. To this dispersed liquid, added was 50 pL of biotin-labelled anti-TSH β antibody (0.75 mg/mL) dissolved in PBS buffer (0.01 M phosphate buffer, 0.0027 M potassium chloride, 0.137 M sodium chloride, pH 7.4). This was subjected to upside-down mixing at room temperature for 15 minutes. The Therma-Max LSA Streptavidin was allowed to aggregate by heating the micro tube to 42° C., and then magnetically collected. Then the supernatant was removed, and excessive biotin-labelled anti-TSH beta antibody was separated (B/F separation). To this, added was 250 pL of TBS buffer. Aggregates were then dispersed by cooling. Subsequently, the excessive amount of biotin was added to cover biotin binding sites of the streptavidin, and then excess biotin was separated (B/F separation). This was further dispersed in PBS buffer (pH 7.4) solution containing 0.5% (w/v) BSA (Sigma), 0.5% (w/v) Tween® 20, and 10 mM EDTA to obtain a first bound substance.

### Preparation of Second Bound Substance

First, 1 mL of an antibody (clone: Mouse 176, mouse IgG, Leinco Technology, Inc., 1 mg/mL), which is a second affinity substance, against human thyroid stimulating hormone (TSH), which is a detection target, was added to 6 mg of 2-mercaptoethanol, and allowed to react at 37° C. for 120 minutes. After the reaction, this was dialyzed against 500 mL of PBS buffer in a dialysis cassette Slide-A-Lyzer (product name) with 10 K MWCO (Pierce) to remove excess 2-mercaptoethanol, and then concentrated to 0.5 mL with ultrafiltration membrane (MILLIPORE [Amicon Ultra-4-Ultracel 100 k]) with a critical exclusion molecular weight of 10000 to obtain an reduced antibody of mouse anti-TSH a antibody. This reduced antibody in an amount of 0.5 mL was allowed to react with 100 pL of maleimidated sodium polyacrylate (33 mg was dissolved in 1 mL of PBS buffer) overnight at 4° C. Subsequently, gel filtration was performed using a Superdex-200 10/300GL (GE Healthcare) to prepare a labelled antibody. The labelled antibody (this antibody is also referred to as a polyacrylated anti-TSH a antibody bound substance) was diluted with an aqueous solution of 0.5% (w/v) BSA (Sigma), 0.5% (w/v) Tween® 20/PBS (pH 7.4), and 10 mM EDTA to a protein concentration of 4 μg/mL, thereby preparing a second bound substance.

It is noted that the above maleimidated sodium polyacrylate was prepared as follows. First, in a 100 mL three-necked flask having a nitrogen gas introducing tube, a thermometer, and a stirrer attached, 2 g of acrylic acid (Wako Pure Chemical Industries, Ltd.), 0.021 g of 2-aminoethanethiol (Wako Pure Chemical Industries, Ltd.), and 0.023 g of azobisisobutyronitrile (Wako Pure Chemical Industries, Ltd.) were dissolved in 50 mL of N,N-dimethylformamide, and purged with nitrogen for 1 hour. Subsequently, a polymerization reaction was performed at 70° C. for 7 hours. The resulting reaction liquid was concentrated to 10 mL under reduced pressure, and re-precipitated with diethyl ether until the viscous material became powdery. White precipitates were filtered, and further dried overnight in a vacuum dryer to obtain amino group-terminated polyacrylic acid (yield: 1.5 g). Next, the amino group-terminated polyacrylic acid was maleimidated. To a 50 mL eggplant flask having a nitrogen gas introducing tube and a stirrer attached, charged and dissolved were 0.5 g of the amino group-terminated polyacrylic acid and 10 mL of N,N-dimethylformamide. To this, 3 mg of EMCS (N-(6-maleimidocaproyloxy)succinimide) (Dojindo Laboratories) was added, and allowed to react overnight. The resulting reaction liquid was concentrated to 1 mL under reduced pressure, and re-precipitated with diethyl ether until the viscous material became powdery. White precipitates were filtered, and further dried overnight in a vacuum dryer to obtain maleimide group-terminated polyacrylic acid. The molecular weight was about 130000 (Tosoh Corp., TSKgel Super AW3000, 6 mm ID. ×150 mm, mobile phase 0.1 M sodium nitrate), and the yield was 0.4 g.

### Sample Preparation

Human thyroid stimulating hormone (TSH; Aspen Bio Pharma, Inc., activity: 8.5 IU/mg, WHO80/558) was dissolved in PBS buffer (pH 7.4) to give a concentration of 30 μg/mL. This solution was diluted to 1 ng/mL with a VITROS TSH calibrator 1 (TSH: 0 mIU/L, Ortho Clinical Diagnostics) to obtain a sample.

### Mixing

The first bound substance in an amount of 150 μL and the second bound substance in an amount of 120 μL were poured into a micro tube, and agitated with a vortex mixer for 1 second. To this micro tube, 750 μL of the above sample was added, and again agitated with a vortex mixer for 60 seconds to obtain a liquid mixture. Similarly, a liquid mixture in which human thyroid stimulating hormone was not present was also prepared as a negative control.

### Measurement

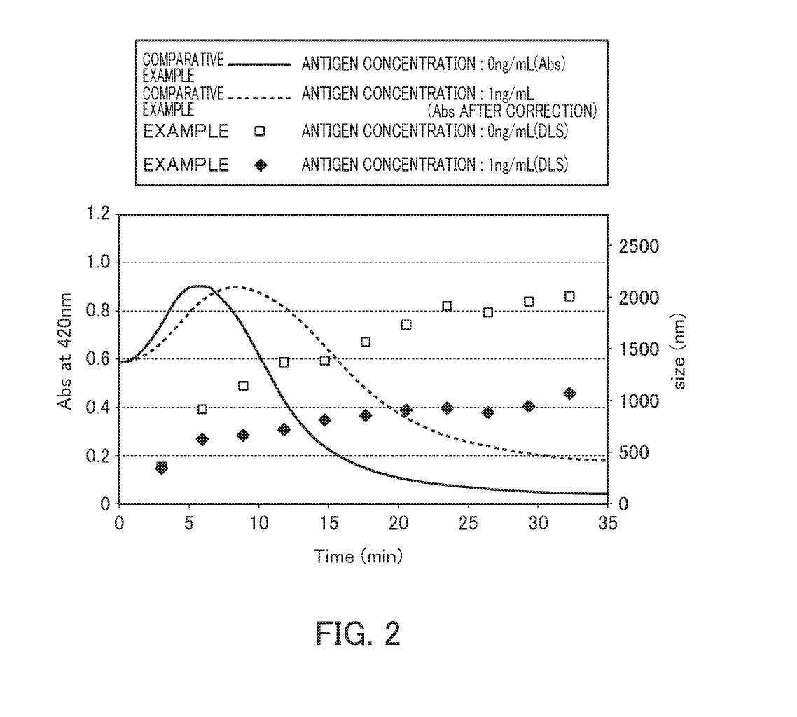

The above liquid mixture was placed in a particle size distribution measuring device by the dynamic light scattering method (DLS) [Malvarn ZETA SIZER Nano-ZS], and the particle size was measured for 35 minutes under aggregation conditions at 37 degrees. Similarly, the particle size was also measured for a liquid mixture used as a negative control. Results are shown in FIG. 2. The instrumental conditions are as follows. Instrumental conditions: Refractive index=1.45, Absorption=0.010

### COMPARATIVE EXAMPLE

### Preparation of First Bound Substance

First, an antibody (clone: Mouse 195, mouse IgG, Leinco Technology, Inc.), which is a first affinity substance, against human thyroid stimulating hormone (TSH), which is a detection target, was biotinylated by the well-known conventional sulfo-NHS-biotin method (AGC Techno Glass Co., Ltd.) to prepare a biotin-labelled anti-TSH beta antibody. Meanwhile, 250 μL of Magnabeat Therma-Max LSA Streptavidin (0.4 mass %) as a streptavidin-attached particulate magnetic substance was transferred into a 1.5 mL micro tube. The Therma-Max LSA Streptavidin was allowed to aggregate by heating the micro tube to 42° C., and then magnetically collected. The supernatant was removed. To this, added was 250 pL of TBS buffer (20 mM Tris-HCI, 150 mM NaCl, pH 7.5). Aggregates were then dispersed by cooling. To this dispersed liquid, added was 50 μL of biotin-labelled anti-TSH β antibody (0.75 mg/mL) dissolved in PBS buffer (0.01 M phosphate buffer, 0.0027 M potassium chloride, 0.137 M sodium chloride, pH 7.4). This was subjected to upside-down mixing at room temperature for 15 minutes. The Therma-Max LSA Streptavidin was allowed to aggregate by heating the micro tube to 42° C., and then magnetically collected. Then the supernatant was removed, and excessive biotin-labelled anti-TSH beta antibody was separated (B/F separation). To this, added was 250 μL of TBS buffer. Aggregates were then dispersed by cooling. Subsequently, the excessive amount of biotin was added to cover biotin binding sites of the streptavidin, and then excess biotin was separated (B/F separation). This was further dispersed in PBS buffer (pH 7.4) solution containing 0.5% (w/v) BSA (Sigma), 0.5% (w/v) Tween® 20, and 10 mM EDTA to obtain a first bound substance.

### Preparation of Second Bound Substance

The second bound substance was prepared as in the method of preparing a second bound substance in Example 1.

### Sample Preparation

Human thyroid stimulating hormone (TSH; Aspen Bio Pharma, Inc., activity: 8.5 IU/mg, WHO80/558) was dissolved in PBS buffer (pH 7.4) to give a concentration of 30 μg/mL. This solution was diluted to 1 ng/mL with a VITROS TSH calibrator 1 (TSH: 0 mlU/L, Ortho Clinical Diagnostics) to obtain a sample.

### Mixing

The first bound substance in an amount of 150 μL and the second bound substance in an amount of 120 μL were poured into a micro tube, and agitated with a vortex mixer for 1 second. To this micro tube, 750 μL of the above sample was added, and again agitated with a vortex mixer for 60 seconds to obtain a liquid mixture. Similarly, a liquid mixture in which human thyroid stimulating hormone was not present was also prepared as a negative control.

### Measurement

A neodymium permanent magnet with dimensions of 5 mm×9 mm×2 mm (Seiko Sangyo Co., Ltd) was attached outside the optical path of a common semi-microcell for a spectrophotometer. The cell was placed in a visible-ultraviolet spectrophotometer “UV-3101PC” (Shimadzu Corporation) equipped with a cell-temperature controller, and was held at 37° C. for 10 minutes or longer.

The above liquid mixture was dispensed into the cell, and zero-correction was performed according to the instructions of the spectrophotometer. Then turbidity (absorbance Abs) was continuously measured over 35 minutes with a slit width of 10 mm using light having a wavelength of 420 nm. Similarly, turbidity (absorbance Abs) was also measured for a liquid mixture prepared as a negative control. Results are shown in FIG. 2 together with the results from Example.

### Results

The results shown in FIG. 2 demonstrate that quantification was able to be performed with higher detection sensitivity even in an early stage (for example, within 10 minutes of measurement time) in Example where quantification was performed based on the particle size as compared with Comparative Example where quantification was performed based on turbidity (absorbance). The results also demonstrate that in Example where quantification was performed based on the particle size, a detection target was able to be detected with high sensitivity without using a magnet (without performing solid-liquid separation) as in Comparative Example.

1. A method of quantifying a detection target in a specimen, the method comprising:
a mixing step of mixing the specimen with a particulate carrier carrying a stimuli-responsive substance and a first affinity substance having an affinity for the detection target to prepare a mixture;
a measurement step of measuring a particle size of a suspended substance in the mixture after exposing the mixture to aggregation conditions of the stimuli-responsive substance; and
a determination step of determining the amount of the detection target based on the particle size.

2. The method according to claim 1, which does not comprise a step of performing solid-liquid separation of an aggregate of the particulate carrier contained in the suspended substance prior to the determination step.

3. The method according to claim 1, wherein the particulate carrier comprises a nonmagnetic substance.

4. The method according to claim 1, wherein the measurement is performed by the dynamic light scattering method.

5. The method according to claim 4, wherein the particulate carrier has a mean refractive index of 1.3 or more.

6. The method according to claim 1, wherein the measurement is performed prior to a time point at which a turbidity of a negative control reaches the maximum value after exposed to the aggregation conditions.

7. The method according to claim 1, wherein a bound substance of a hydrophilic substance and a second affinity substance having an affinity for the detection target is further mixed at the mixing step in addition to the particulate carrier.
